(19) United States
(12) Patent Application Publication
(10) Pub. No.: US 2026/0232965 A1
(43) Pub. Date: Aug. 13, 2026

(54) MEDICAL DEVICES, MEDICAL SYSTEMS, AND RELATED METHODS

(71) Applicants: Richard J. LINDER, Sandy, UT (US); Steve KUBOW, Carlsbad, CA (US); XENTER, INC., Draper, UT (US)

(72) Inventors: Richard J. LINDER, Sandy, UT (US); Steve KUBOW, Carlsbad, CA (US)

(21) Appl. No.: 19/152,895

(22) PCT Filed: Feb. 1, 2024
(86) PCT No.: PCT/US2024/014037

§ 371 Date: Jul. 31, 2025

Related U.S. Application Data
(60) Provisional application No. 63/442,982, filed on Feb. 2, 2023.

(51) Int. Cl. A61M 25/06; A61M 25/00; A61M 25/09; A61M 39/06

(52) U.S. Cl. CPC A61M 25/0662; A61M 25/005; A61M 25/09; A61M 39/06; A61M 2025/09175; A61M 2039/0626; A61M 2205/3561; A61M 2205/3592; A61M 2205/50; A61M 2205/82

(57) ABSTRACT

Various embodiments of medical devices, systems and related methods are provided. In one embodiment, a system includes a first elongated member, such as a guidewire, is configured for insertion into a body, the first elongated member having at least a first electronic component associated therewith. A second elongated member, such as a guide catheter, is configured for insertion into a body, the second elongated member having at least a second electronic component associated therewith and positioned adjacent a distal end of the second elongated member. A device is conductively coupled with the at least a second electronic component and configured to provide power thereto, and the at least a second electronic component is configured to generate an electrical field and transmit power to the at least a first electronic component via the electrical field.

### CROSS-REFERENCE TO RELATED APPLICATIONS

This application claims the benefit of and priority to U.S. Provisional Patent Application Ser. No. 63/442,982 filed on Feb. 2, 2023, and entitled “MEDICAL DEVICES, MEDICAL SYSTEMS, AND RELATED METHODS”, the disclosure of which is incorporated herein by reference in its entirety.

### BACKGROUND

The present invention relates generally to medical devices incorporating electrical components, as well as systems and methods incorporating such devices. In one, non-limiting example, such medical devices may include intraluminal devices, such as guidewires and catheters, which include various sensors for measuring one or more physiological parameters and/or for purposes of imaging.

Guidewire devices are often used to lead or to guide catheters or other interventional devices to a targeted anatomical location within a patient's body. Typically, guidewires are passed into and through a patient's vasculature in order to reach the target location, which may be, for example, at or near the patient's heart or brain. Radiographic imaging is typically utilized to assist in navigating a guidewire to the targeted location. Guidewires are available with various outer diameter sizes. Widely utilized sizes include 0.010, 0.014, 0.016, 0.018, 0.024, and 0.035 inches in diameter, for example, though they may also be smaller, larger or other intermediate sizes in diameter.

In many instances, a guidewire is placed within the body during the interventional procedure where it can be used to guide one or more catheters or other interventional devices to the targeted anatomical location. Once in place, a catheter can be used to image the targeting location, to aspirate clots or other occlusions, or to deliver drugs, stents, embolic devices, radiopaque dyes, or other devices or substances in association with diagnosing and/or treating the patient.

These types of interventional devices can include sensors located at the distal end in order to provide added functionality to the device. In one example, a guidewire having a pressure sensor, a flow sensor, or other sensor may be used to help diagnose whether there is a stenosis in a patient's vascular system. In another example, intravascular ultrasound (IVUS) is an imaging technique that may utilize a catheter or other interventional device having an ultrasound imaging sensor attached at or near the distal end. Ultrasound waves are utilized to provide imaging from within targeted vasculature (e.g., the coronary arteries).

There are several challenges associated with using sensors with intraluminal devices. For example, such interventional devices involved have very limited space to work in, given the stringent dimensional constraints involved. Moreover, integrating the sensors with the interventional device in a way that maintains effective functionality can be challenging.

The use of such interventional devices is also challenging due to the conventional need to manage several long lengths of wires and other components, including guidewires, power cables, data wires, and the like. Care must be taken with respect to what is allowed in the sterile field and when it can be removed. Additional staff is often required simply to manage such wires and cables.

As such, there is an ongoing need for improved medical devices that effectively integrate sensors and can help provide data (including imaging) in a more efficient manner and/or provide data not previously obtainable in a practical manner.

### SUMMARY

Embodiments of the present disclosure medical devices, systems, and methods. In some embodiments, a guide catheter includes electrical components and may be configured to facilitate transmission of power to a second device, such as a guidewire, and/or facilitate transmission of data signals to or from electronic components associated with the second device.

In one particular embodiment a system is provided that includes a first elongated member configured for insertion into a body, the first elongated member having at least a first electronic component associated therewith. The system additionally includes a second elongated member configured for insertion into a body, the second elongated member having at least a second electronic component associated therewith and positioned adjacent a distal end of the second elongated member. A device is conductively coupled with the at least a second electronic component and configured to provide power thereto, and the at least a second electronic component is configured to generate an electrical field and transmit power to the at least a first electronic component via the electrical field.

In one embodiment, the first elongated member includes a guidewire.

In one embodiment, the second elongated member includes a guide catheter.

In one embodiment, the guidewire extends through a lumen of the guide catheter.

In one embodiment, the at least a first electronic component is positioned near a distal end of the guidewire.

In one embodiment, the device includes a housing positioned at a proximal end of the guide catheter.

In one embodiment, the device includes a hemostatic valve coupled with the guide catheter.

In one embodiment, the guide catheter includes a structural layer.

In one embodiment, the structural layer comprises metal braiding.

In one embodiment, the metal braiding is conductively coupled with the at least a second electronic component.

In one embodiment, the system further comprises a hemostatic valve fluidly coupled with the guide catheter.

In one embodiment, the at least one electronic component includes at least one of a sensor and an application specific integrated circuit.

In another embodiment, a guide catheter is provided that comprises a body having a fluid passage defined therethrough, a connecting structure at a proximal end of body, at least one electronic component disposed at or near a distal end of the body; and at least one conductor electrically coupled with the at least one electronic component and extending toward the proximal end of the body.

In one embodiment the device further comprises a conductive coupling member associated with the connecting structure.

In one embodiment the device further comprises a housing associated with the connecting structure, wherein a control unit is disposed within the housing.

In one embodiment, the control unit includes a processor and a power source.

In one embodiment, the control unit includes a wireless transceiver.

In one embodiment, the at least one electronic component includes a device configured to generate an electrical field.

In one embodiment, the at least one conductor comprises a metallic structural layer of the body.

In one embodiment, the first connecting structure includes a Luer lock structure.

In yet another embodiment, a method is provided of electrically connecting medical devices. The method comprises providing a guide catheter, providing a guidewire, generating an electrical field at a distal end of the guide catheter, and transmitting power to an electronic component associated with the guidewire via the electrical field.

In one embodiment the method further comprises transmitting data from the electronic component to a control unit via the electrical field.

In one embodiment the method further comprises configuring a structural layer of the guidewire to perform at least one of the following: generate the electrical field; transmit power from a proximal end of the guide catheter to the distal end of the guide catheter; and transmit a data signal between a distal end of the guide catheter to a proximal end of the guide catheter.

It is noted that the variously recited embodiments are not to be limited and that features, aspects, components and acts of one described embodiment may be combined with features, aspects, components or acts of another described embodiment.

### BRIEF DESCRIPTION OF THE SEVERAL VIEWS OF THE DRAWINGS

The foregoing and other advantages of the embodiments of the present disclosure will become apparent upon reading the following detailed description and upon reference to the drawings in which:

FIG. 1 illustrates a medical device system according to an embodiment of the present disclosure;

FIG. 2 illustrates a guide catheter according to one embodiment of the present disclosure;

FIG. 3 illustrates a partial cross-section of the guide catheter as indicated by section lines 3-3 shown in FIG. 2;

FIG. 4 illustrates an enlarged detail of a portion the guide catheter illustrated in FIG. 2;

FIG. 5 illustrates a medical device system according to one embodiment of the present disclosure; and FIG. 6 illustrates a medical device system according to another embodiment of the present disclosure.

### DETAILED DESCRIPTION

Various embodiments described herein are directed toward the incorporation of electronic devices (e.g., sensors and transducers) into medical devices and systems and the transfer of power and data to and from such electronic devices.

In some embodiments, devices associated with cardiovascular, neurovascular, and endovascular procedures are provided having sensors or other electric components integrated therewith. For example, guidewires or catheters may include sensors, transducers or other electronic or optical components integrated into the structure for detecting or measuring physiological data (e.g., pressure, flow rate, etc.), providing imaging data (e.g., ultrasound images), and provide that data in real time during an associated procedure.

In some embodiments, other sensors or electronic elements are associated with the device. For example, sensors configured to detect the presence of biological components may be incorporated into or otherwise associated with the device. In some embodiments, an antenna structure may be integrated with the device for providing wireless transmission of data.

Some non-limiting examples of medical devices that may incorporate such sensors include those described in: U.S. patent application Ser. No. 17/205,964, entitled “Guidewire for Imaging and Measurement of Pressure and Other Physiological Parameters” and filed on Mar. 18, 2021; U.S. patent application Ser. No. 17/205,854, entitled “Catheter for Imaging and Measurement of Pressure and Other Physiological Parameters” and filed on Mar. 19, 2021; U.S. patent application Ser. No. 17/205,754, entitled “Operatively Coupled Data and Power Transfer Device for Medical Guidewires and Catheters with Sensor” and filed on Mar. 18, 2021; U.S. patent application Ser. No. 17/205,614, entitled “Signal Conducting Device for Concurrent Power and Data Transfer to and From Un-wired Sensors Attached to a Medical Device” and filed on Mar. 18, 2021; U.S. patent application Ser. No. 17/979,629, entitled “Data and Power Transfer Devices for Use with Medical Devices and Related Methods” and filed Nov. 2, 2022; and U.S. Provisional Patent Application No. 63/394,591, entitled “Medical Devices, Systems, and Methods Including Power and Data Transfer” and filed Aug. 2, 2022, the disclosures of which are each incorporated by reference herein in their entireties

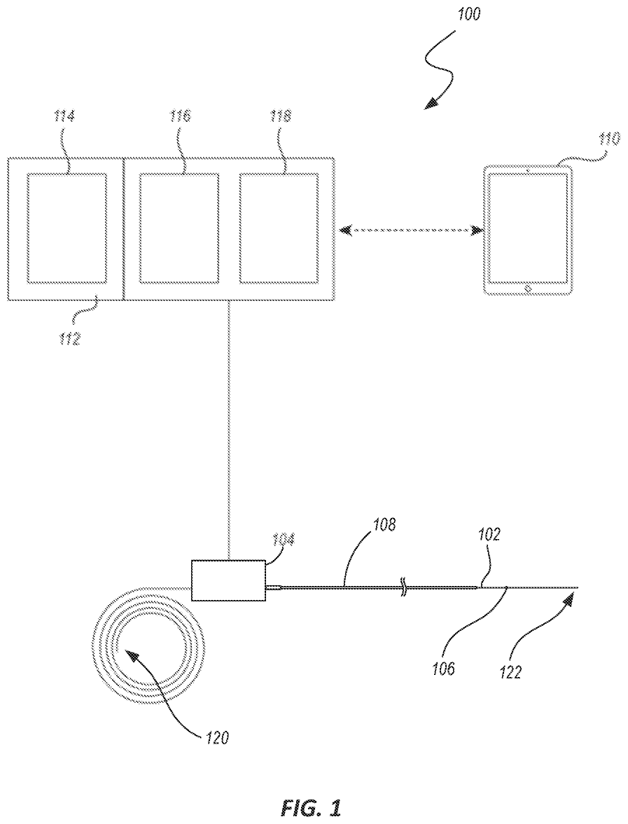

Referring to FIG. 1, a medical device system 100 is illustrated according to an embodiment of the present disclosure. As shown, the system 100 includes a guidewire device 102, a proximal device 104, one or more sensors 106 associated with a the guidewire device 102, a guide catheter 108 (sometimes called a guiding catheter), a control unit 112 (shown enlarged and in schematic form, at least a portion of which may be incorporated physically into the proximal device 104 as will be described in association with various embodiments below) and an external device 110 (e.g., a stationary, portable, or handheld computer; a stationary, portable, or handheld display; a tablet computer; a smart phone; or other input and/or output device). The control unit 112 includes a power source 114, data signal processor 116, and optionally a transceiver (a transmitter and/or receiver) 118, which may be in wired or wireless communication with the external device 110.

When used in wireless communications, the transceiver 118 may be configured to communicate using any of a variety of protocols or technologies including, without limitation, Bluetooth®, Wi-fi, Zigbee, or other appropriate communications technologies.

The proximal device 104 may be configured as a data and/or power transfer device. For example, the proximal device 104 may be configured for electrical coupling with the guidewire device 102 to provide power to sensors (e.g., sensors 106) or other electronics associated with the guidewire device 102 and/or to receive various signals provided by the sensors or other electronic components. Such electrical coupling between the proximal device 104 and the guidewire device 102 may be accomplished via direct conductive contact or via an electronic field (e.g., through a capacitive coupling or inductive coupling).

The data signal processor 116 may be configured to provide instructions to electronic components of the guidewire device 102 and/or to receive sensor data signals sent through the guidewire device 102 from one or more sensors 106 associated with the guidewire device 102. The power source 114 may be configured to transmit power through the guidewire device 102 to power the one or more sensors 106 and/or other components of the guidewire device 102. The power source 114 may include an on-board power source, such as a battery or battery pack, and/or may include a wired connection to an external power source.

The one or more sensors 106 may be located at any suitable position on the guidewire device 102 but may often be disposed at the distal section which is expected to reach the targeted anatomy. As used herein, the “distal section” or “distal portion” refers to the distal-most 30 cm of the device, the distal-most 20 cm of the device, the distal-most 15 cm of the device, the distal-most 10 cm of the device, or to a range using any two of the foregoing values as endpoints. In some embodiments, the “intermediate section” may be considered generally as the section between the proximal section and the proximal section. In some embodiments the intermediate section may be considered as roughly the middle third of the device. The “proximal section” or “proximal portion” may be considered as roughly the proximal-most third of the device.

In one embodiment, the system 100 is configured to send power to the sensors 106 through individual traces or other electrical conductors, and data signals from the sensors may be transmitted via separate transmission structures or schemes. For example, data may be transmitted by additional traces or electrical conductors, by optical transmission, or by wireless transmission.

In another embodiment, the system 100 is configured to send power and data signals through a structural component of the guidewire device 102 (e.g., through a core wire or a hypo tube) itself rather than through traces or through separate, discrete electrical conductors. In some embodiments, multiple power and/or data signals (e.g., data signals from multiple sensors) can be sent through the guidewire device 102 (e.g., the structural component or one or more dedicated electrical conductors) simultaneously. Power and/or data signals can also be sent in a “continuous” fashion. That is, the power and/or data signals can have a sufficiently high sampling rate such that the information is provided to the user within time frames that are practically “real-time” and are perceived as such by a user of the system or device. Nonlimiting examples of managing power and/or data signals, along with associated components and systems, as set forth in the previously incorporated U.S. patent applications.

The guidewire device 102 of the system 100 is configured for insertion into the body of the subject. The subject is typically a human, but in other implementations may be a non-human mammal or even non-mammalian animal. Any suitable route of administration may be utilized, depending on particular preferences and/or application needs. Common routes include femoral, radial, and jugular, but the system 100 may utilize other access routes as needed.

The guidewire device 102 has a proximal end 120 and a distal end 122. The length of the guidewire device 102 may vary according to particular application needs and targeted anatomical area. As an example, the guidewire device 102 may have an overall length from proximal end 120 to distal end 122 of about 50 cm to about 350 cm, more commonly about 200 cm, depending on particular application needs and/or particular anatomical targets. The guidewire device 102 may have a size such that the outer diameter (e.g., after application of other outer members such as sleeves and/or coatings) is about 0.008 inches to about 0.040 inches, though larger or smaller sizes may also be utilized depending on particular application needs. For example, particular embodiments may have outer diameter sizes corresponding to standard or nominal guidewire sizes such as 0.010 inches, 0.014 inches, 0.016 inches, 0.018 inches, 0.024 inches, 0.035 inches, 0.038 inches, or other such sizes common to guidewire devices. In some embodiments, the guidewire may exhibit an outer cross-sectional dimension (e.g., a diameter) within a range using any of the above dimensions as an endpoint. The guidewire device 102 may be formed from stainless steel or other metal or alloy having similar appropriate properties. In some embodiments, the guidewire device 102 may be formed of or may comprise a conductive material of appropriate mechanical properties.

One or more sensors 106 of the system 100 may include a pressure sensor, a flow sensor, an imaging sensor, a component detection sensor (e.g., for detection of a biological component), or combinations thereof, for example. Additionally, while generally referred to as a “sensor” in discussing various embodiments throughout, such “sensors” may comprise or otherwise be associated with transducers or other electrical, electromechanical, electrochemical, or optical components and may be configured as input devices, output devices, or both. In other words, sensor components described herein may include or be a part of transducers that are capable of transmitting and/or receiving a specified type of signal. For example, such sensors may include ultrasound transducers, optical emitters (e.g., light emitting diodes (LEDs)), optical sensors (e.g., photo diodes) and the like.

As previously noted, the one or more sensors may additionally, or alternatively, be configured to sense the presence of biological components or measure physiological parameters in the targeted anatomical location (e.g., in the blood). Example biological components that may be detected/measured include sugar levels, pH levels, CO2 levels (CO2 partial pressure, bicarbonate levels), oxygen levels (oxygen partial pressure, oxygen saturation), temperature, and other such substrates and physiological parameters. The one or more sensors may be configured to sense the presence, absence, or levels of biological components such as, for example, immune system-related molecules (e.g., macrophages, lymphocytes, T cells, natural killer cells, monocytes, other white blood cells, etc.), inflammatory markers (e.g., C-reactive protein, procalcitonin, amyloid A, cytokines, alpha-1-acid glycoprotein, ceruloplasmin, hepcidin, haptoglobin, etc.), platelets, hemoglobin, ammonia, creatinine, bilirubin, homocysteine, albumin, lactate, pyruvate, ketone bodies, ion and/or nutrient levels (e.g., glucose, urea, chloride, sodium, potassium, calcium, iron/ferritin, copper, zinc, magnesium, vitamins, etc.), hormones (e.g., estradiol, follicle-stimulating hormone, aldosterone, progesterone, luteinizing hormone, testosterone, thyroxine, thyrotropin, parathyroid hormone, insulin, glucagon, cortisol, prolactin, etc.), enzymes (e.g., amylase, lactate dehydrogenase, lipase, creatine kinase), lipids (e.g., triglycerides, HDL cholesterol, LDL cholesterol), tumor markers (e.g., alpha fetoprotein, beta human chorionic gonadotrophin, carcinoembryonic antigen, prostate specific antigen, calcitonin), and/or toxins (e.g., lead, ethanol).

In the embodiment shown in FIG. 1 (which is not to be considered a to-scale representation), multiple sensors 106 may arranged longitudinally along a length of a distal portion of the guidewire device 102. In one embodiment, the sensors 106 are configured as pressure sensors (e.g., a capacitive-type sensor or a piezo-type sensor). In addition to any sensor positioned in the distal portion of the guidewire device 102, one or more sensors may be located in the proximal portion of the wire, and/or one or more sensors may be located in the intermediate section of the guidewire device 102.

While not specifically shown, a coil and/or atraumatic tip may be located at the distal end 122 of the guidewire device 102 as will be appreciated by those of skill in the art. The coil may be a single coil or multiple connected or interwoven coils. Additionally, or alternatively, a polymer material may be positioned on or applied to all or a portion (e.g., the distal section of the guidewire device 102). The atraumatic tip may include a sphere, partial sphere, or other curved geometrical configuration to protect against potential trauma that might otherwise be caused by the distal end of the guidewire device 102. The atraumatic tip may be formed from a polymer adhesive material and/or solder, for example.

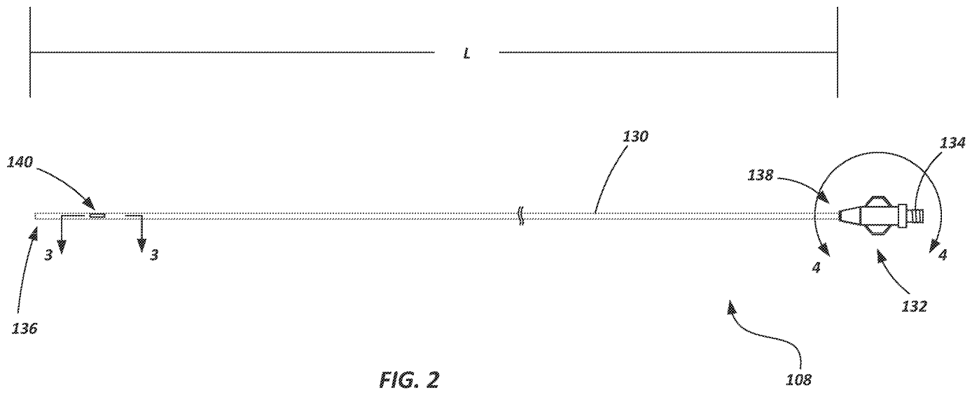

Referring to FIGS. 2-4, a guide catheter 108 is shown. The guide catheter 108 includes an elongated, flexible body 130 coupled with a connector portion 132. The connector portion 132 may include, for example, a female or male portion of a Luer lock connection 134. In other embodiments, the connector portion 132 may include a connector other than a Luer lock type connector including any of various threaded, compression, or other mechanical type connectors. The guide catheter 108 includes one or more lumens defined through its longitudinal extent for passage of the guidewire device 102 and/or other devices.

The body 130 may include, or be constructed from, multiple layers. For example, in one embodiment, the body 130 may include a lubricious polytetrafluoroethylene (PTFE) inner layer, a reinforcement layer, and an outer soft nylon elastomer jacket. In one embodiment, the reinforcement layer may include a braided stainless-steel material used to stiffen the catheter, providing support for passage of the body 108 through a patient's vessel or other anatomy. In some embodiments, in order to achieve a reduced outer diameter, the reinforcement layer and the outer layer may be integrated into a single composite or hybrid layer.

The guide catheter 108 may exhibit a variety of lengths. In one example, the body 130 of the guide catheter 108 may exhibit a length L from its distal end 136 to its proximal end 138 of approximately 100 centimeters (cm). In other embodiments, the length L may be less than, or greater than, 100 centimeters, and may range, for example, from 50 cm to approximately 150 cm. In another embodiment, the length L may range from approximately 90 cm to approximately 125 cm. Of course, other lengths may be exhibited by the body 130 of the guide catheter 108.

Similarly, the body 130 of the guide catheter 108 may exhibit various diameters. For example, the guide catheter 108 may be classified as a 5F (French), a 6F, or a 7F catheter (with 1 French being equal to approximately 0.33 millimeter (mm)—and, therefore, with a 5F catheter exhibiting a diameter of approximately 1.66 mm). Of course, the body 130 of the guide catheter 108 may exhibit larger or smaller diameters.

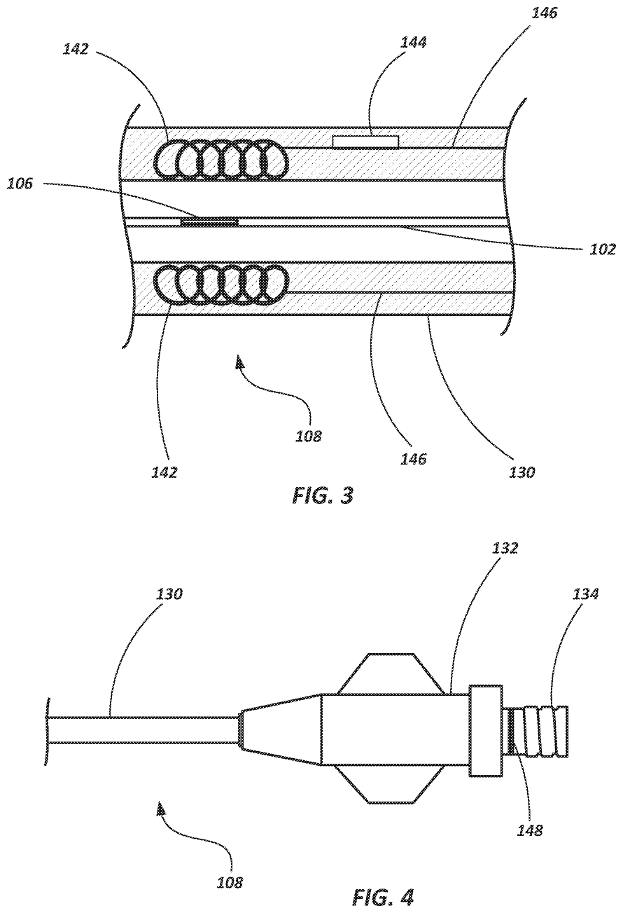

In one embodiment, the guide catheter 108 may include at least one electrical component 140 at or near the distal end 136 of the body 130. Such an electrical component, or electrical components, may include, for example, a device 142 for generating an electrical field or for contactless transmission of data or power (e.g., via inductive or capacitive transfer). It is noted that, in FIG. 3, the device 142 for generating an electrical field is shown schematically as a pair of inductor coils. However, any number of inductor coils may be used. In another embodiment, a capacitive plate (e.g., configured as a ring, or otherwise) may be used as the device 142 rather than an inductive member.

Additionally, the electrical component(s) 140 may optionally include a processor 144 or other electrical circuit device including, for example, an application specific integrated circuit (ASIC) device. Such a component may be used in facilitating power distribution or communication of data with an associated guidewire (or other medical device, such as another catheter) as shall be described in further detail herein below.

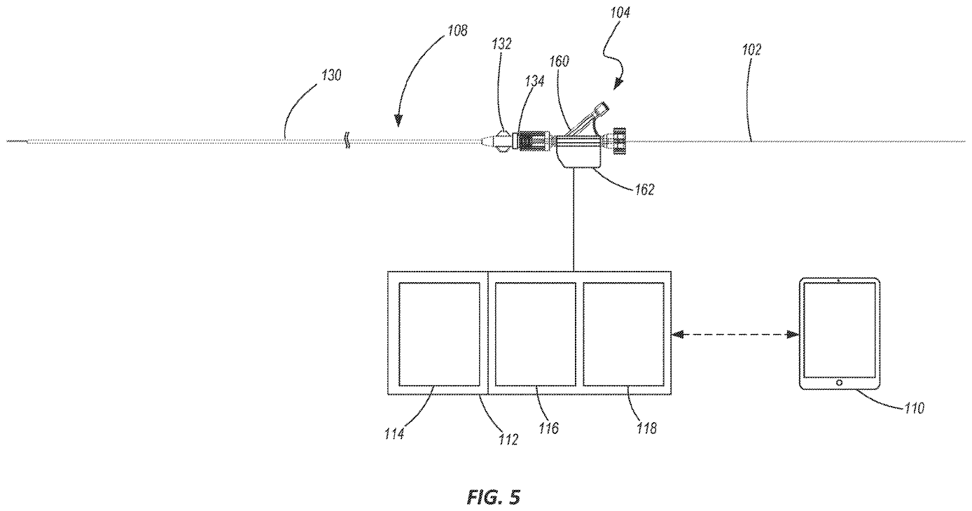

The electrical component(s) 140 may be coupled to one or more wires, conductive traces (e.g., associated with a flexible membrane), or other conductive members (referred to generally as conductors 146 herein). In the embodiment shown in FIG. 2, such conductors 146 may extend to the proximal end 138 of the body 130 and be electrically and physically coupled with a conductive pad or other conductive coupling member 148. For example, the conductive coupling member 148 may include a conductive ring or pad that is located on or otherwise associated with the connector portion 132 (see FIG. 4). The conductive coupling member 148 may, for example, be located within the connector 134 (e.g., the Luer lock connector) for physical and electrical coupling with another device (e.g., for coupling with a similar conductive coupling member associated with a proximal device 104).

In some embodiments, the electrical component(s) 140 may be additionally, or alternatively coupled, (electrically and physically) with the structural layer of the body 130. For example, when the structural layer includes a metal braid, the metal braid may be used for transmitting power and/or data to and from the electrical component(s) 140. In another embodiment, the metal braid (or other structural member) may be used in a generating the electrical field. In another embodiment, the metal braid (or other structural member) may be used to provide a ground path to the circuit formed by the various members and components. Thus, the structural layer of the body 130 may be used as a power path, a data signal path, a ground path, as an electrical field generator, or some combination thereof.

The electrical components (e.g., electrical field generating device 142) may be configured to transmit power to the guidewire device 102 (shown in conjunction with the partial cross-sectional view of FIG. 3), to transmit instructional signals to the guidewire, and/or to receive data signals from the guidewire device 102. In some embodiments, the electrical field generating device may use an inductive field or a capacitive field to facilitate transmission of power and/or data to and/or from a conductive member of the guidewire device 102 such as described and detailed in the previously incorporated documents. As previously noted, in some embodiments, such a conductive member may include a structural member of the guidewire device 102 such as a core wire or a hypo tube. In some embodiments, the electrical field may be in communication with an electric circuit (e.g., an ASIC), which is then in communication with sensors 106 and other electrical components associated with the guidewire device 102. The electrical field may maintain communication with the guidewire device 102 even while the guidewire device 102 is being physically translated relative to the guide catheter 108.

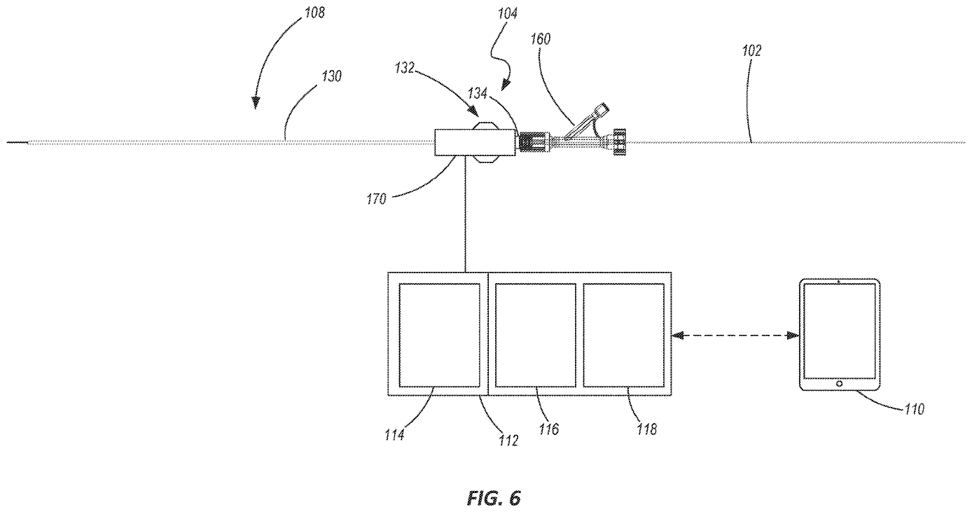

Referring now to FIG. 5 (with continued reference to FIGS. 1-4), an embodiment of a medical device system 100 according to one embodiment of the present disclosure is shown in further detail. The system 100 is generally similar to that described with respect to FIG. 1 and shows a proximal device 104 that includes both a hemostatic valve 160 (e.g., a Touhy valve) and a data/power transmission device 162. In one embodiment, the valve 160 and data/power device 162 may comprise an integrated device such that a common body or housing 164 incorporates both the valve components and the control unit 112 with its associated components (e.g., components 114, 116, 118). It is noted that, as with FIG. 1 (and FIG. 6 to be discussed below), the control unit 112 is shown in an enlarged, schematic form and in actual construction, may be physically integrated into the proximal device-in this case the data/power transmission device 162.

An electrical connection is formed between the proximal device 104 and the connection portion 130 of the guide catheter 108 such as by way of the conductive coupling member 148 (see FIG. 4) via a mating conductive coupling member formed in the proximal device 104 as discussed in further detail below.

The system 100 may then function such as described above in association with FIG. 1. Power and data may be transmitted such as described above and as detailed in previously incorporated documents. Moving the electric field (e.g., inductive or capacitive) to a location within the guide catheter 108, such as a location near the distal end 136 of the body 130, enables enhanced transmission efficiencies and integrity. In some embodiments, use of the guide catheter 108 for generation of the electric field may reduce the physical distance that power and data signals have to travel (between electrical components of the guidewire device 102 and the electrical field generated by the guide catheter 108) to a distance of approximately 20 cm or less, approximately 10 cm or less, approximately 5 cm or less, or even 1 cm or less.

In another embodiment, the data and power transfer device 162 and the hemostatic valve 160 may be discrete components that selectively couple with each other, both electrically and physically. Examples of such an embodiment are described in previously incorporated U.S. patent application Ser. No. 17/979,629, entitled “Data and Power Transfer Devices for Use with Medical Devices and Related Methods.”

Referring now to FIG. 6, an embodiment of a medical device system 100 according to one embodiment of the present disclosure is shown in further detail. The system 100 is generally similar to that described with respect to FIG. 1 and includes a proximal device 104 and associated control unit 112 that is integrated into a housing 170 that may serve as, or otherwise be associated with, the connector portion 132 of the guide catheter 108. A hemostatic valve 160 may be coupled with the guide catheter 108, but such may include a conventional valve that does not include components associated with the control unit 112 (given that the control unit is associated with the connector portion of the guide catheter in the presently described embodiment). When used with a conventional hemostatic valve 160 that does not include electronic components, a conductive coupling member 148 may not be required in association with the connector portion 132. However, in some embodiments, the hemostatic valve 160 may still have components (e.g., a pressure sensor) associated with it, and the conductive coupling member 148 may still be desirable for communication between any component associated with the hemostatic valve 160 and the control unit which may be disposed with or otherwise associated with the housing 170.

In yet another embodiment, a power and transfer device may be a discrete component (relative to the guide catheter) and be selectively coupled with housing 170 or other member of the guide catheter 108 in a manner similar to that which was described above with respect to FIG. 5 (although the connection is made with the housing 170 or other portion of the guide catheter 108 rather than the hemostatic valve 160).

The use of “adapted to” or “configured to” herein is meant as open and inclusive language that does not foreclose devices adapted to or configured to perform additional tasks or acts. Additionally, the use of “based on” is meant to be open and inclusive, in that a process, act, calculation, or other action “based on” one or more recited conditions or values may, in practice, be based on additional conditions or values beyond those recited. Headings, lists, and numbering included herein are for ease of explanation only and are not meant to be limiting.

When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition.

It will be understood that components and/or features described in the context of one embodiment may also be included in any other embodiment disclosed herein, unless specified otherwise.

While the disclosed embodiments may be susceptible to various modifications and alternative forms, specific embodiments have been shown by way of example in the drawings and have been described in detail herein. It is noted that features, elements, or components of one embodiment may be combined with features, elements, or components of other embodiments without limitation. Thus, it should be understood that the invention is not intended to be limited to the particular forms disclosed. Rather, the invention includes all modifications, equivalents, and alternatives falling within the spirit and scope of the disclosure as defined by the following appended claims.

What is claimed is:

1. A system comprising:
a first elongated member configured for insertion into a body, the first elongated member having at least a first electronic component associated therewith;
a second elongated member configured for insertion into a body, the second elongated member having at least a second electronic component associated therewith and positioned adjacent a distal end of the second elongated member;
a device conductively coupled with the at least a second electronic component and configured to provide power thereto, wherein the at least a second electronic component is configured to generate an electrical field and transmit power to the at least a first electronic component via the electrical field.

2. The system of claim 1, wherein the first elongated member includes a guidewire.

3. The system of claim 2, wherein the second elongated member includes a guide catheter.

4. The system of claim 3, wherein the guidewire extends through a lumen of the guide catheter.

5. The system of claim 3, wherein the at least a first electronic component is positioned near a distal end of the guidewire.

6. The system of claim 5, wherein the device includes a housing positioned at a proximal end of the guide catheter.

7. The system of claim 5, wherein the device includes a hemostatic valve coupled with the guide catheter.

8. The system of claim 3, wherein the guide catheter includes a structural layer.

9. The system of claim 8, wherein the structural layer comprises metal braiding.

10. The system of claim 9, wherein the metal braiding is conductively coupled with the at least a second electronic component.

11. The system of claim 3, further comprising a hemostatic valve fluidly coupled with the guide catheter.

12. The system of claim 1, wherein the at least one electronic component includes at least one of a sensor and an application specific integrated circuit.

13. A guide catheter comprising:
a body having a fluid passage defined therethrough;
a connecting structure at a proximal end of body;
at least one electronic component disposed at or near a distal end of the body;
at least one conductor electrically coupled with the at least one electronic component and extending toward the proximal end of the body.

14. The guide catheter of claim 13, further comprising a conductive coupling member associated with the connecting structure.

15. The guide catheter of claim 13, further comprising a housing associated with the connecting structure, wherein a control unit is disposed within the housing.

16. The guide catheter of claim 15, wherein the control unit includes a processor and a power source.

17. The guide catheter of claim 16, wherein the control unit includes a wireless transceiver.

18. The guide catheter of claim 13, wherein the at least one electronic component includes a device configured to generate an electrical field.

19. The guide catheter of claim 13, wherein the at least one conductor comprises a metallic structural layer of the body.

20. The guide catheter of claim 15, wherein the first connecting structure includes a Luer lock structure.

21. A method of electrically connecting medical devices, the method comprising:
providing a guide catheter;
providing a guidewire;
generating an electrical field at a distal end of the guide catheter;
transmitting power to an electronic component associated with the guidewire via the electrical field.

22. The method according to claim 21, further comprising transmitting data from the electronic component to a control unit via the electrical field.

23. The method according to claim 22, further comprising configuring a structural layer of the guidewire to perform at least one of the following:
generate the electrical field;
transmit power from a proximal end of the guide catheter to the distal end of the guide catheter; and
transmit a data signal between a distal end of the guide catheter to a proximal end of the guide catheter.
